(19) United States
(12) Plant Patent Application Publication
(10) Pub. No.: US 2016/0366798 P1
(43) Pub. Date: Dec. 15, 2016

(54) Raspberry plant named 'SAN RAFAEL'

(50) ;

(71) Applicant: Juan Enrique Castillo, Sevilla (ES)

(72) Inventor: Juan Enrique Castillo, Sevilla (ES)

(21) Appl. No.: 14/545,736

(22) Filed: Jun. 11, 2015

(51) Int. Cl. A01H 5/00

(52) U.S. Cl. PLT/204

(57) ABSTRACT

A new raspberry plant named ‘SAN RAFAEL’ is disclosed. The plants of “SAN RAFAEL” are characterized by the late maturity of fruit, strong shoot growth, and fruiting buds covering the entire length of the fruiting canes.

### CROSS-REFERENCE TO RELATED APPLICATIONS

None

### STATEMENT REGARDING FEDERALLY SPONSORED RESEARCH OR DEVELOPMENT

None

### GENUS AND SPECIES

Rubus idaeus L.

### VARIETY DENOMINATION

‘SAN RAFAEL’

### BACKGROUND OF THE INVENTION

‘SAN RAFAEL’ is a product of a controlled breeding program carried out by the inventor in the Geria, in the Andalucia region of Spain. Prior to 2014, the original single plant of ‘SAN RAFAEL’ was expanded to multiple plants, which were planted in the area of Geria, Spain. By 2014, the plants had been observed and evaluated for several years. Throughout several generations of asexual propagation, ‘SAN RAFAEL’ has been observed to retain its distinctive characteristics and remain true to type.

### BRIEF DESCRIPTION OF THE PHOTOGRAPHS

FIG. 1 illustrates fruit in various stages of maturity of a mature ‘SAN RAFAEL’ raspberry plant;

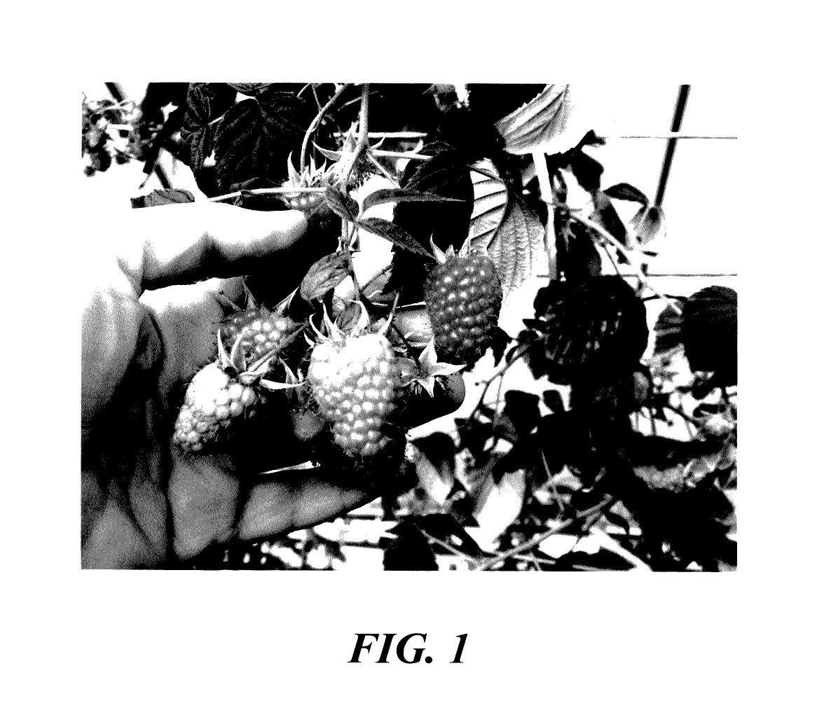

FIG. 2 illustrates a mature ‘SAN RAFAEL’ raspberry plant with fruit buds;

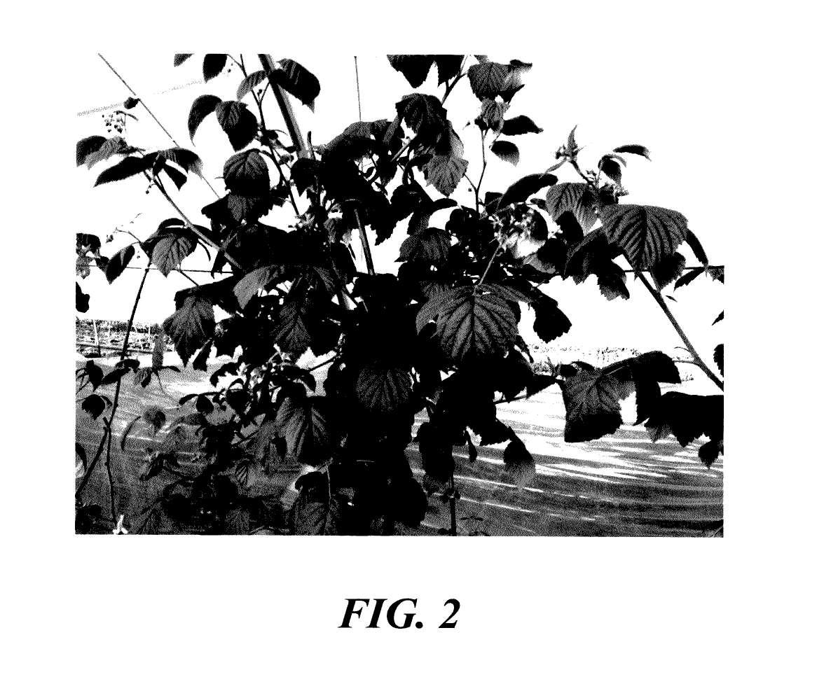

FIG. 3 illustrates a leaf of a mature ‘SAN RAFAEL’ raspberry plant;

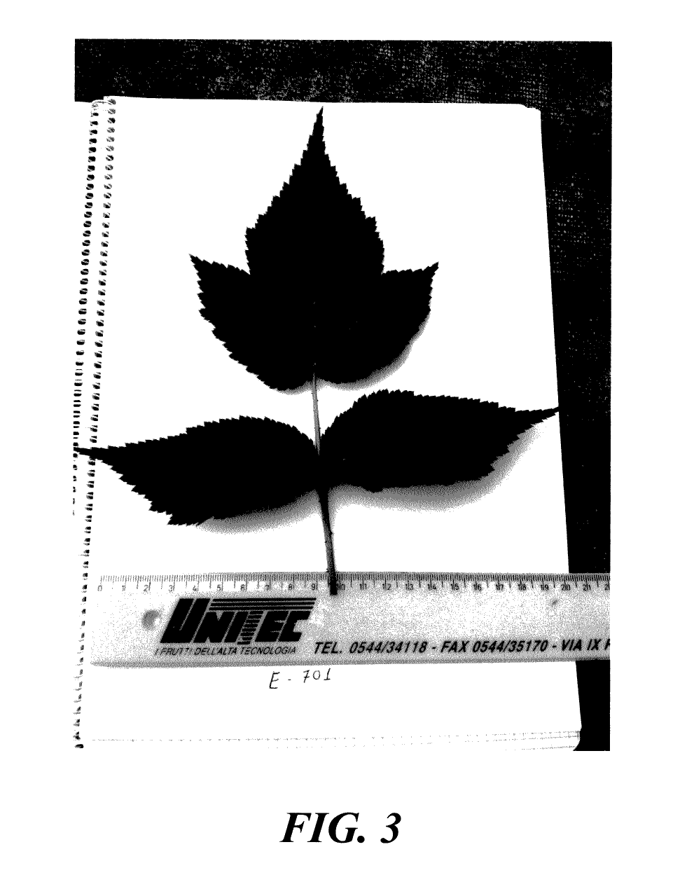

FIG. 4 illustrates flowers of a mature ‘SAN RAFAEL’ raspberry plant;

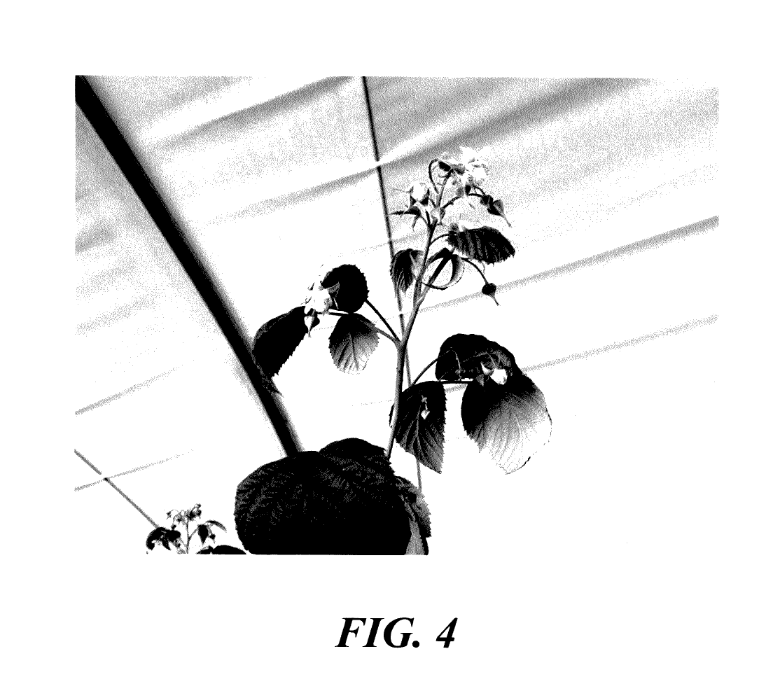

FIG. 5 illustrates vines of mature ‘SAN RAFAEL’ raspberry plants growing on a trellis system; and

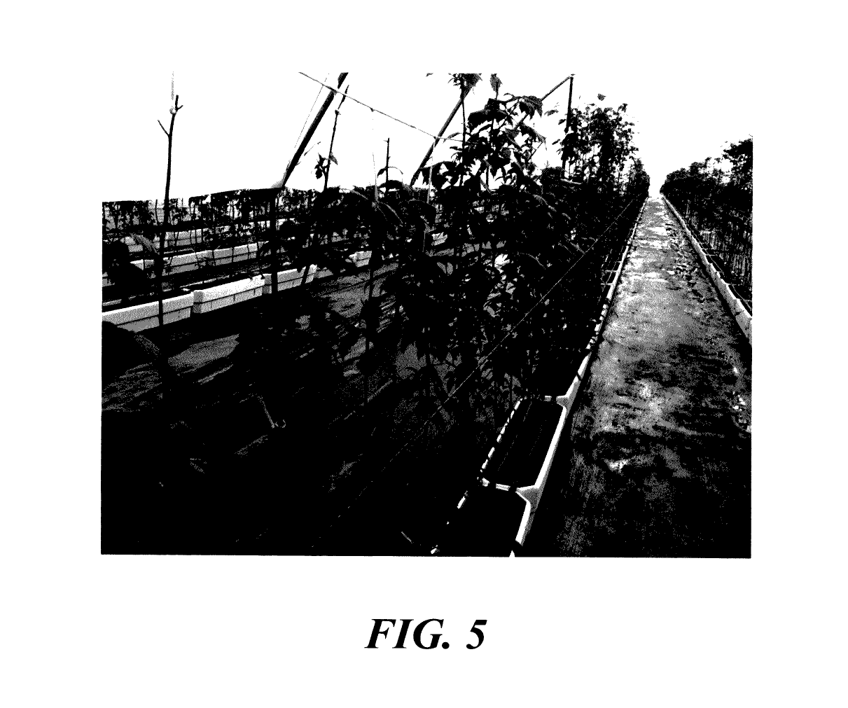

FIG. 6. Illustrates fully mature, harvested fruit from ‘SAN RAFAEL’ raspberry plants.

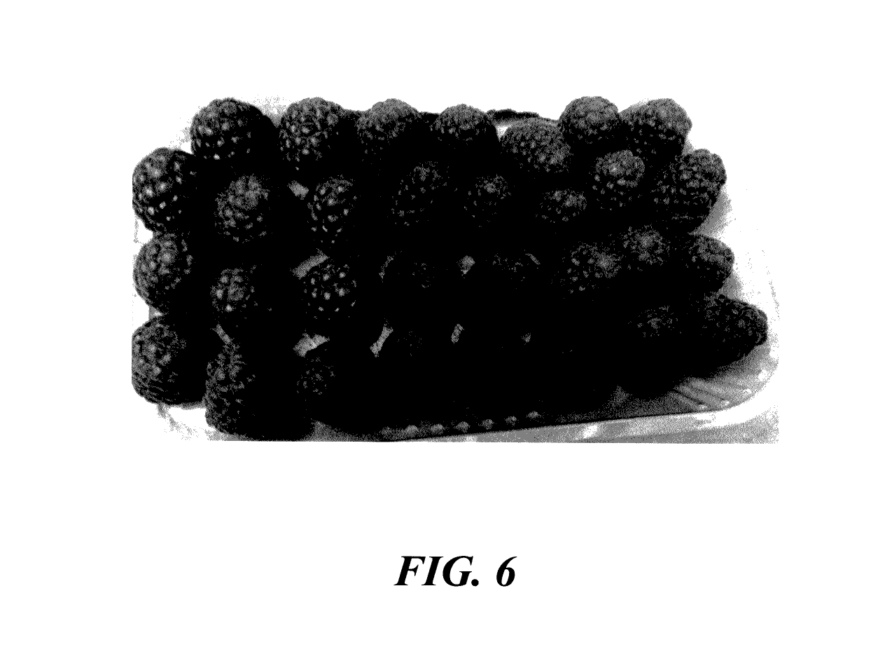

The colors of these illustrations may vary with lighting conditions and, therefore, color characteristics of this new variety should be determined with reference to the observations described herein, rather than from these illustrations alone.

### DETAILED BOTANICAL DESCRIPTION

The following description is based on observations made during the 2013 and 2014 growing seasons in the Andalucia region of Spain. It should be understood that the characteristics described will vary somewhat depending upon cultural practices and climatic conditions, and can vary with location and season. Quantified measurements are expressed as an average of measurements taken from a number of individual plants of the new variety.

The measurements of any individual plant or any group of plants, of the new variety may vary from the stated average. All color references are based on The Royal Horticultural Society Color Chart.
Use: To produce raspberry fruit for the fresh and processed markets.
Number of current season's canes: Medium (similar to Glen Ample, Multiraspa, and Rumiloba)
Anthocyanin coloration of apex during rapid growth of very young shoot: This characteristic is absent (similar to Gelbe Antwerpener)
Spines present: Yes (similar to Malling Promise)
Fruit ratio (length/width): Medium to Large (in between Malling Delight/Tulameen and Glen Cove/Rafzter)
General fruit shape in lateral view: Trapezoidal (similar to Gradina)
Fruit color: Medium red (similar to Glen Clova, Mailing Orion)
Fruit bearing characteristics: Fruit is present only on previous season's cane in the summer (similar to Mailing Promise)
Time of beginning of fruit ripening on previous season's canes: Late (similar to Mailing Landmark, Schönemann)

What is claimed is:

1. A new and distinct raspberry plant as shown and described herein.
